(19) United States
(12) Patent Application Publication
(10) Pub. No.: US 2026/0217659 A1
(43) Pub. Date: Jul. 30, 2026

(54) COMPOUNDS, COMPOSITIONS AND USES THEREOF IN THE TREATMENT AND PREVENTION OF DISEASES AND CONDITIONS ASSOCIATE WITH OR AGGRAVATED BY IMPAIRED MITOPHAGY OR OXIDATIVE STRESS

(71) Applicant: Yissum Research Development Company of The Hebrew University of Jerusalem Ltd., Jerusalem (IL)

(72) Inventors: Shmuel BEN-SASSON Z"L, Jerusalem (IL); Einav GROSS, Jerusalem (IL)

(73) Assignee: Yissum Research Development Company of The Hebrew University of Jerusalem Ltd., Jerusalem (IL)

(21) Appl. No.: 19/145,052

(22) PCT Filed: Jan. 4, 2024
(86) PCT No.: PCT/IL2024/050018

§ 371 Date: Jul. 1, 2025

Related U.S. Application Data
(60) Provisional application No. 63/478,360, filed on Jan. 4, 2023.

(51) Int. Cl. C07D 233/84; A61K 31/137; A61K 31/4178; A61K 31/4192; A61K 31/4196; A61P 35/00; C07C 211/27; C07D 249/04; C07D 249/12

(52) U.S. Cl. CPC C07D 233/84; A61K 31/137; A61K 31/4178; A61K 31/4192; A61K 31/4196; A61P 35/00; C07C 211/27; C07D 249/04; C07D 249/12

(57) ABSTRACT

The invention provides 1,2-derivated phenylene compounds and composition comprising them and uses thereof in the method of treatment and prevention of diseases and conditions associated with or aggravated by impaired mitophagy or oxidative stress.

### BACKGROUND OF THE INVENTION

Mitochondria (MT) are double-membrane-bound organelles found in most eukaryotic organisms. They are essential for chemical energy production, in the form of ATP, in all aerobic organisms, including humans. Moreover, mitochondria are essential for many other metabolic processes, including the synthesis of amino acids, lipids, heme, steroid hormones, and are the source of reactive oxygen species (ROS).

ROS present cells with a double-edged sword. On the one hand, they play a crucial role in many cellular and physiological processes, including the innate immune response and the degradation and recycling of the cellular milieu in a process called autophagy. On the other hand, ROS interact with metals to produce toxic oxygen (O₂) radicals that can damage DNA and biological membranes, thus interfering with mitochondrial function and cause cell injury and death. Mitochondria generate ROS as part of their physiological activity and are especially vulnerable to ROS-induced damage. Therefore, to maintain healthy mitochondria, there is a constant need to generate new mitochondrial components (mitochondrial biogenesis) while removing the damaged ones through mitophagy (=mitochondrial-autophagy).

The proper functioning of this intracellular “quality-control” mechanism of mitophagy is vital in tissues where no renewal by cell division is taking place. Cells of nonrenewable tissues include neurons, skeletal muscle, heart muscle cells, insulin-producing beta-cells of the endocrine pancreas, retinal pigment epithelium cells, and more. Indeed, degenerative diseases associated with aging belong mainly to such nonrenewable tissue, including dementia, Alzheimer's and Parkinson's diseases, sarcopenia (=skeletal muscle atrophy), congestive heart failure, type 2 diabetes, age-related macular degeneration, fibrosis, including lung-fibrosis, and more. In addition, various cancers whose incidence also increases with age are also typified by impaired mitophagy (for a review see Perwez A et al. Parkin: A targetable linchpin in human malignancies. Biochim Biophys Acta—Reviews on Cancer (2021), 1876:188533 and Friedlander J E et al. Failure to Gard: Mitochondrial Protein Quality Control in Cancer. Int. J. Mol. Sci. (2021), 22:8306).

While mitochondria biogenesis does not generally decline with age (and may even increase), mitophagy is profoundly decreased. Therefore, accumulating damaged mitochondria is thought to underlie the decline in organ function and health span. The current consensus is that impaired mitophagy plays a pivotal role in developing these degenerative diseases associated with aging (Markaki M. et al. Int Rev Cell Mol Biol (2018) 340:169-208).

It was shown that subjects with Parkinson's disease (PD) have compromised mitophagy processes (Lee S H et al. (2016) EMBO Mol Med 8:779-85; Gao F. et al. Frot Neurol (2017) 8:527). Indeed, mitochondrial dysfunction appears to be a key factor in the pathophysiology of both familial and sporadic PD, as well as in cases of toxin-induced Parkinsonism (Rayn B J. et al. Trends Biochem Sci (2015) 40:200-10).

Inadequate mitophagy leads to excessive ROS formation. Therefore, mitophagy links oxidative stress conditions and neurodegenerative diseases (Shefa U. et al. Neural Regen Res (2019) 14:749-756). Thus, the terms “impaired mitophagy” and “oxidative stress” or “oxidative injury” are hereby used interchangeably to describe unfavorable conditions that lead to the evolvement of aging-associated diseases, including cancer.

The toxin 1-methyl 4-phenyl 1,2,3,6-tetrahydropyridine (MPTP) induces Parkinsonian syndrome in people (Langston W J. et al. Science (1983) 219:979-980). Since the chemical structures MPTP and the pesticide N,N′-dimethyl-4,4′-bipyridinium dichloride (paraquat) are similar, paraquat is widely used in animal models of PD (Miller G W. Toxicol Sci (2007) 100:1-2). Moreover, paraquat (PQ) is a robust inducer of oxidative stress in cells (Halliwell B., Gutteridge J. in Free Radicals in Biology and Medicine, Clarendon Press, Oxford, 2006.). Therefore, induction of mitophagy should increase the resistance to PQ-induced oxidative injury. In agreement with the above, it was shown that compromising C. elegans mitophagy makes this organism more vulnerable to PQ toxicity (Luz A L et al. Toxicology (2017) 387:81-94).

Thus, it is well established that protecting cells and organisms against paraquat-induced damage is a hallmark of mitophagy augmentation (Dagda R A et al. Int. J. Mol. Sci. 14:22163-89 (2013)).

There is a need for medicaments and methods capable of treating and/or protecting the human body from the damages of impaired mitophagy/mitochondrial-autophagy and oxidative injury, including any conditions, diseases, disorders, and symptoms associated therewith and also including conditions and diseases associated with cell degeneration, in particular in cells of non-regenerative tissues and various cancers.

### SUMMARY OF THE INVENTION

The present invention provides a compound having a general formula (I);

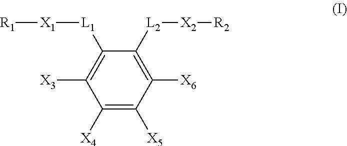

wherein R₁ and R₂ are each independently selected from —C(═NR₃)NR₄R₅, —NR₆R₇, —N⁺R₈R₉R₁₀, —NR₁₁C(═N)NR₁₂R₁₃, —NR₁₈NR₁₉R₂₀, —NR₁₄C(═N)—NR₁₅—C(═N)—NR₁₆R₁₇═N—R₂₁

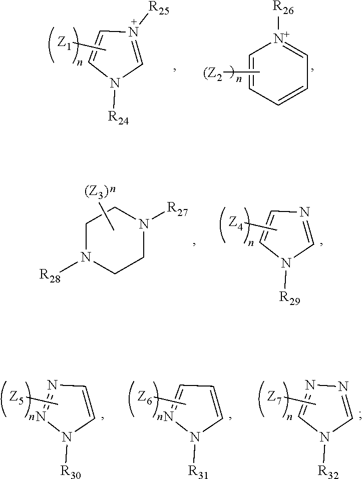

wherein each of R₃-R₃₂ is independently selected from H, straight or branched C₁-C₁₂ alkyl, straight or branched C₂-C₁₂ alkenyl, straight or branched C₂-C₁₂ alkynyl, phenyl, —OH and any combinations thereof; each of L₁ and L₂ is independently selected from straight or branched C₄-C₁₂ alkylene, straight or branched C₄-C₁₂ alkenylene, straight or branched C₄-C₁₂ alkynylene; each L₁ and L₂ is independently optionally interrupted by at least one of C₄-C₈ cycloalkylene, C₄-C₈ cycloalkenylene, C₄-C₈ cycloalkynylene, arylene, heteroarylene, heteroatom and any combinations thereof; each of L₁ and L₂ is independently optionally substituted with at least one of halogen and any combinations thereof; each of X₁ and X₂ is independently selected from null (i.e. L₁ and R₁ and/or L₂ and R₂ are directly connected) —O—, —S—, —S(═O)—, —S(═O)₂—; each of Z₁-Z₇ is independently a halogen; wherein n is independently an integer selected from 0-8; each of X₃—X₆ is independently selected from H, halogen (F, Br, I, C₁), astatine (At), tennessine (Ts) and any combinations thereof.

In some embodiments, when X¹ and X₂ is each selected from —O—, —S— than each of R₁ and R₂ is independently selected from

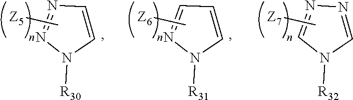

In some embodiments, L is straight or branched C₄-C₁₂ alkylene. In some embodiments, L is straight or branched C₄-C₈ alkylene. In some embodiments, L is straight or branched C₄-C₁₂ alkylene. In some embodiments, L is straight or branched C₁₀-C₁₂ alkylene. In some embodiments, L is straight or branched C₄ alkylene. In some embodiments, L is straight or branched C₅ alkylene. In some embodiments, L is straight or branched C₆ alkylene. In some embodiments, L is straight or branched C₇ alkylene. In some embodiments, L is straight or branched C₈ alkylene. In some embodiments, L is straight or branched C₉ alkylene. In some embodiments, L is straight or branched C₁₀ alkylene. In some embodiments, L is straight or branched C₁₁ alkylene. In some embodiments, L is straight or branched C₁₂ alkylene.

The invention further provides provides a compound having a general formula (I);

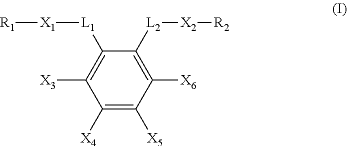

wherein R₁ and R₂ are each independently selected from —C(═NR₃)NR₄R₅, —NR₆R₇, —N⁺R₈R₉R₁₀, —NR₁₁C(═N)NR₁₂R₁₃, —NR₁₈NR₁₉R₂₀, —NR₁₄C(═N)—NR₁₅—C(═N)—NR₁₆R₁₇═N—R₂₁

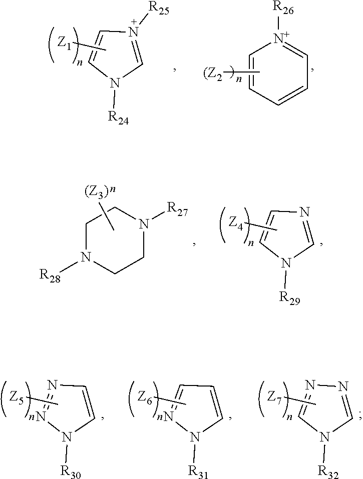

wherein each of R₃-R₃₂ is independently selected from H, straight or branched C₁-C₁₂ alkyl, straight or branched C₂-C₁₂ alkenyl, straight or branched C₂-C₁₂ alkynyl, phenyl, —OH and any combinations thereof; each of L₁ and L₂ is independently selected from straight or branched C₄-C₁₂ alkylene, straight or branched C₄-C₁₂ alkenylene, straight or branched C₄-C₁₂ alkynylene; each L₁ and L₂ is independently optionally interrupted by at least one of C₄-C₈ cycloalkylene, C₄-C₈ cycloalkenylene, C₄-C₈ cycloalkynylene, arylene, heteroarylene, heteroatom and any combinations thereof; each of L₁ and L₂ is independently optionally substituted with at least one of halogen and any combinations thereof; each of X₁ and X₂ is independently selected from —O—, —S—, —S(═O)—, —S(═O)₂—; each of Z₁-Z₇ is independently a halogen; wherein n is independently an integer selected from 0-8; each of X₃—X₆ is independently selected from H, halogen (F, Br, I, Cl), astatine (At), tennessine (Ts) and any combinations thereof.

The invention further provides a pharmaceutical composition comprising a compound of formula (I) as defined herein above.

It should be understood that the term “interrupted by” as used herein refers to the option wherein at least one moiety as listed herein above is connected between any two carbon atoms of L, thus said at least one moiety has two open valencies. Furthermore, the term “substituted with” should be understood to relate to the option of substituting at least one hydrogen atom of L with at least one moiety as listed herein above, thus said at least one moiety has one open valency.

In some embodiments, L is interrupted by at least one of C₄-C₈ cycloalkylene, C₄-C₈ cycloalkenylene, C₄-C₈ cycloalkynylene, aryl, heteroaryl, heteroatom and any combinations thereof. In other embodiments, L is interrupted by at least one C₄-C₈ cycloalkylene. In further embodiments, L is interrupted by at least one C₄-C₈ cycloalkenylene. In some embodiments, L is interrupted by at least one C₄-C₈ cycloalkynylene. In some embodiments, L is interrupted by at least one aryl selected from phenyl or biphenyl. In some embodiments, L is interrupted by at least one heteroaryl. In some embodiments, L is interrupted by at least one heteroatom selected from N, O, S. In some embodiments, L is substituted with at least one of halogen selected from F, Br, Cl, I, and any combinations thereof.

In some embodiments, R₁ and R₂ are identical. In other embodiments, R₁ and R₂ are different. In some embodiments, X₁ and X₂ are identical. In other embodiments, X₁ and X₂ are different.

In some embodiments, at least one of X₁ and X₂ is null. In some embodiments, each of X₁ and X₂ is independently selected from —O—, —S—, —S(═O)—, —S(═O)₂. In some embodiments, at least one of X₁ and X₂ is-O—. In some embodiments, at least one of X₁ and X₂ is-S—. In some embodiments, at least one of X₁ and X₂ is-S(═O)—. In some embodiments, at least one of X₁ and X₂ is-S(═O)₂.

In some embodiments, R₁ and R₂ are each —C(═NR₃)NR₄R₅. In some embodiments, R₁ and R₂ are each selected from —NR₆R₇ and —N+R₈R₉R₁₀. In some embodiments, R₁ and R₂ are each selected from —NR₁₁C(═N)NR₁₂R₁₃ and —NR₁₄C(═N)—NR₁₅—C(═N)—NR₁₆R₁₇. In some embodiments, R₁ and R₂ are each —NR₁₈NR₁₉R₂₀. In some embodiments, R₁ and R₂ are each ═N—R₂₁. In some embodiments, R₁ and R₂ are each

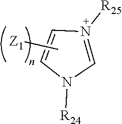

wherein R₂₄, R₂₅ and Z₁ are as defined herein above, and n is selected from 0-3. In some embodiments, R₁ and R₂ are each

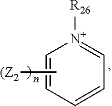

wherein R₂₆ and Z₂ are as defined herein above, and n is selected from 0-5. In some embodiments, R₁ and R₂ are each

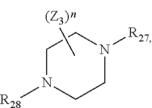

wherein R₂₇, R₂₆ and Z₃ are as defined herein above, and n is selected from 0-8. In some embodiments, R₁ and R₂ are each

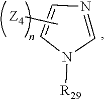

wherein R₂₉ and Z₄ are as defined herein above, and n is selected from 0-4. In some embodiments, R₁ and R₂ are each

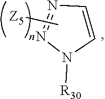

wherein R₃₀ and Z₅ are as defined herein above, and n is selected from 0-4. In some embodiments, R₁ and R₂ are each

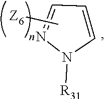

wherein R₃₁ and Z₆ are as defined herein above, and n is selected from 0-4. In some embodiments, R₁ and R₂ are each

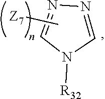

wherein R₃₂ and Z₇ are as defined herein above, and n is selected from 0-4.

In some embodiments each n is 1. In other embodiments, each n is 2. In other embodiments each n is selected from 0, 1, 2, 3, 4, 5, 6, 7, 8. When referring to n being 0 it should be understood that said Z₁, Z₂, Z₃, Z₄, Z₅, Z₆ or Z₇ is not substituted on a ring and thus H is placed on each open valency of the ring as required by the ring structure. When referring to n being 1 it should be understood that said Z₁, Z₂, Z₃, Z₄, Z₅, Z₆ or Z₇ is substituted once on a ring and that H is placed on each open valency of the ring as required by the ring structure. When referring to n being 2 it should be understood that said Z₁, Z₂, Z₃, Z₄, Z₅, Z₆ or Z₇ is substituted twice on a ring and that H is placed on each open valency of the ring as required by the ring structure. When referring to n being 3 it should be understood that said Z₁, Z₂, Z₃, Z₄, Z₅, Z₆ or Z₇ is substituted three times on a ring, if and where possible as required by the ring structure and that H is placed on each open valency of the ring as required by the ring structure.

In some embodiments, X₁ and X₂ are each independently selected from —O—, —S— and any combinations thereof. In some embodiments, X₁ and X₂ are each independently selected —S(═O)—, —S(═O)₂— and any combinations thereof. In further embodiments each of X₁ and X₂ is selected from —S(═O)— and —S(═O)₂—.

In some embodiments, each of Z₁, Z₂, Z₃, Z₄, Z₅, Z₆ and Z₇ independently is a halogen. In some embodiments, Z₁ is a halogen. In some embodiments, Z₂ is a halogen. In some embodiments, Z₃ is a halogen. In some embodiments, Z₄ is a halogen. In some embodiments, Z₅ is a halogen. In some embodiments, Z₆ is a halogen. In some embodiments, Z₇ is a halogen. In some embodiments, said halogen is F. In other embodiments, said halogen is selected from F, Cl, Br and I.

In some other embodiments, R₁ and R₂ are each selected from

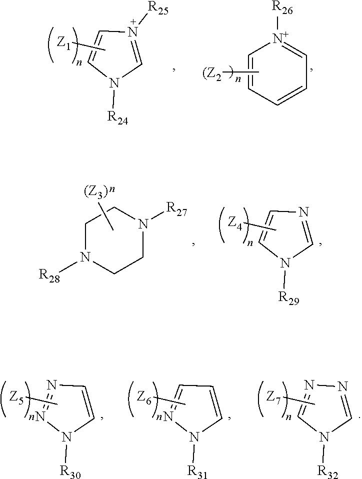

In further embodiments at least one of X₃—X₆ is H. In further embodiments at least one of X₃—X₆ is different than H. In further embodiments at least one of X₃—X₆ is halogen. In further embodiments at least one of X₃—X₆ is astatine. In further embodiments at least one of X₃—X₆ is tennessine.

In some embodiments, a compound of the invention is selected from:

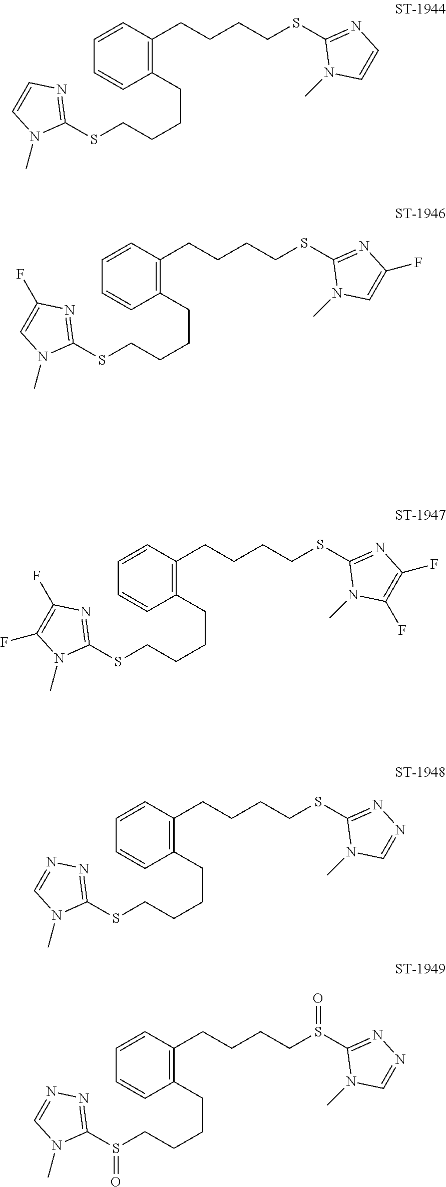 [SMILES: Cn1ccnc1SCCCCc1ccccc1CCCCSc1nccn1C ; Cn1cc(F)nc1SCCCCc1ccccc1CCCCSc1nc(F)cn1C ; Cn1c(SCCCCc2ccccc2CCCCSc2nc(F)c(F)n2C)nc(F)c1F ; Cn1cnnc1SCCCCc1ccccc1CCCCSc1nncn1C ; Cn1cnnc1S(=O)CCCCc1ccccc1CCCCS(=O)c1nncn1C]

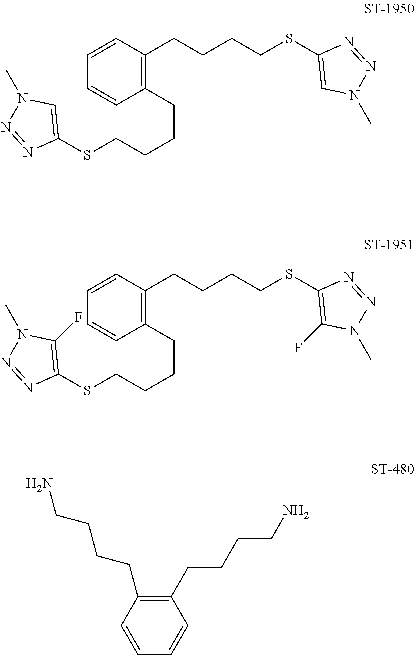 [SMILES: Cn1cc(SCCCCc2ccccc2CCCCSc2cn(C)nn2)nn1 ; Cn1nnc(SCCCCc2ccccc2CCCCSc2nnn(C)c2F)c1F ; NCCCCc1ccccc1CCCCN]

In another aspect the invention provides a composition comprising at least one compound as defined herein above.

In some embodiments, said composition further comprises at least one further pharmaceutically active agent. In some embodiments, at least one further pharmaceutically active agent is at least one lysosome inhibitor or an autophagy inhibitor. In some embodiments said at least one lysosome inhibitor is selected from chloroquine, hydroxychloroquine, bafilomycin A, ammonium chloride, LysO5, leupeptide, pepstatin A, E64d, 3-methyladenine, monesin, liensinine and any combinations thereof.

In further aspect, the invention provides a compound as defined herein above, for use it the treatment of a condition or a disease associated with cell degeneration, including cancer.

When referring to “treatment of a disease, disorder, symptom, which is caused by, associated with, or aggravated by impaired mitophagy” it should be understood to encompass the management and care of a patient to combat disease, disorder, condition or symptom and includes the slowing the progression or delaying of the progression of the disease, disorder, condition or symptom, the alleviation or relief of symptoms and complications, and/or the cure or elimination of the disease, disorder or condition. Said condition, disease, disorder, or symptom are defined to be associated with directly or indirectly, caused by directly or indirectly or directly or indirectly aggravated by impaired mitophagy process, i.e., the cellular process of removing damaged mitochondria is biologically inefficient, reduced, and insufficient for maintaining a healthy viable cell. In some embodiments, the mitophagy process is a process in cells of non-regenerative tissues.

When referring to “prevention of a disease, disorder, symptom, which is caused by, associated with, or aggravated by impaired mitophagy” it should be understood to encompass substantially stopping the occurrence or progression of a disease, disorder, condition, or symptom. Said condition, disease, disorder or symptom are defined to be associated with directly or indirectly, caused by directly or indirectly or directly or indirectly aggravated by impaired mitophagy process, i.e., the cellular process of removing damaged mitochondria is biologically inefficient, reduced, and insufficient for maintaining a healthy viable cell. In some embodiments, the mitophagy process is a process in cells of non-regenerative tissues.

When relating to the use of compounds of the invention in the “treatment of a condition, disease, disorder or symptom associated with cell degeneration”, it should be understood to relate to the management and care of a patient to combat a disease, disorder, condition or symptom and includes the prevention or delaying of the progression of the disease, disorder, condition or symptom, the alleviation or relief of symptoms and complications, and/or the cure or elimination of the disease, disorder or condition. Said condition, disease, disorder, or symptom are defined to be associated with, caused by, or aggravated by the process of inexorable slide into no functionality of cells caused by stochastic degradation of its parts, in some embodiments, the mitochondria. In further embodiments, the invention is directed to the treatment of conditions, disorders, diseases or symptoms associated with cell degeneration of non-regenerative tissues and cancer. Such “non-regenerative tissue” includes tissues that do not spontaneously regenerate, such as neurons (central and peripheral nervous system), cardiomyocytes (heart muscle cells), skeletal-muscle cells, insulin-producing cells (beta-cells of the endocrine pancreas), and retinal pigment epithelium. In addition, the invention is directed to the treatment of cancer, a disease known to be associated with impaired mitophagy and distorted metabolism.

In a further aspect, the invention provides a compound, as defined herein above, for use it the slowing the progression of or preventing a condition or a disease associated with cell degeneration, including cancer.

When referring to “slowing the progression” it should be understood to relate to delaying of the progression of the disease, disorder, condition or symptom, associated with, caused by, or aggravated by cell degeneration, including cancer. In some embodiments, the invention is directed to treating conditions, disorders, diseases or symptoms associated with cell degeneration of non-regenerative tissue and cancer.

When referring to “preventing” it should be understood to substantially stop the occurrence or progression of the disease, disorder, condition, or symptom associated with, caused by, or aggravated by cell degeneration. In some embodiments, the invention is directed to treating conditions, disorders, diseases or symptoms associated with cell degeneration of non-regenerative tissue and cancer.

In some embodiments, said condition or a disease associated with cell degeneration is a neurodegenerative disease, disorder, and condition associated therewith.

Yet, in some other embodiments, said condition or a disease associated with cell degeneration is cancer.

In other embodiments, said condition or a disease associated with cell degeneration is an age-related disease, disorder, and condition associated therewith.

In I further embodiments, said condition or a disease associated with cell degeneration is selected from Parkinson's disease, Alzheimer's disease, dementia, congestive heart failure, sarcopenia, type 2 diabetes, age-related macular degeneration (AMD), atherosclerosis, cardiovascular diseases, cancer, liver diseases, pancreatic diseases, ocular diseases, arthritis, cataracts, osteoporosis, hypertension, fibrosis, including lung-fibrosis, and any combinations thereof.

The invention further provides a compound as defined herein above and below for use in a method of maintaining the vitality of non-regenerating tissue in a subject, said method comprising administering to said subject an effective dose of a compound as defined herein above and below.

When referring to “maintaining the vitality of non-regenerating tissue” it should be understood to relate to keeping the vital state of a non-regenerating tissue by slowing down the progression or preventing said tissue cell degeneration. Upon maintaining the vitality of non-regenerative tissue, the lifespan of a subject treated with a compound of the invention can be prolonged.

The invention further provides a method of maintaining the vitality of non-regenerating tissue in a subject, said method comprising administering to said subject an effective dose of a compound as defined herein above and below.

The invention further provides a method for the treatment of a condition, or a disease associated with cell degeneration in a subject, said method comprising administering to said subject an effective dose of a compound as defined herein above and below.

The invention further provides a method for the treatment of a malignant disease in a subject, said method comprising administering to said subject an effective dose of a compound as defined herein above and below.

The invention further provides a method for slowing the progression of or preventing a condition or a disease associated with cell degeneration in a subject, said method comprising administering to said subject an effective dose of a compound as defined herein above and below.

In further aspect, the invention provides a compound as defined herein above, for use in facilitating mitophagy and preventing oxidative injury. When referring to the facilitation of mitophagy or preventing oxidative injury, it should be understood to encompass the promotion of, enhancement of, and enablement of the process of mitophagy in cells or preventing oxidative injury, thereby prolonging the viability of said cells. In some embodiments, said cells are of non-regenerative tissue.

In further aspect, the invention provides a compound as defined herein above, for use in facilitating mitophagy in cancer cells, in order to promote their death. For example, it was shown that in hepatocellular carcinoma (HCC, known also as liver cancer), triggering mitophagy, will result in enhanced HCC apoptosis (for a brief review see: Aman Y. et al. Iron out, mitophagy in! A way to slow down hepatocellular carcinoma. EMBO Reports (2020) 21: e51652).

The invention further provides a method of reducing the tumor-load of a human cancer, said method comprising administering to said subject an effective dose of a compound of the invention, as disclosed herein above and below.

In some embodiments, said method of reducing the tumor-load of a human cancer further comprises administering to a subject in need thereof, in addition, an effective dose of at least one autophagy inhibiting agent. In some embodiments, said at least one autophagy inhibiting agent is selected from chloroquine and hydroxychloroquine.

In some embodiments of a method or use of the invention, said treatment further comprises administering to a subject in need thereof one further pharmaceutically active agent. In some embodiments, at least one further pharmaceutically active agent is at least one lysosome inhibitor or an autophagy inhibitor. In some embodiments said at least one lysosome inhibitor is selected from chloroquine, hydroxychloroquine, bafilomycin A, ammonium chloride, LysO5, leupeptide, pepstatin A, E64d, 3-methyladenine, monesin, liensinine and any combinations thereof.

The term “straight or branched C₁-C₁₂ alkyl” should be understood to encompass any straight or branched saturated hydrocarbon chain having 1, 2, 3, 4, 5, 6, 7, 8, 9, 10, 11 or 12 carbon atoms, wherein only sigma bonds connect between the atoms of the chain, and wherein one hydrogen atom is removed from any carbon atom of the chain.

The term “straight or branched C₂-C₁₂ alkenyl” should be understood to encompass any straight or branched unsaturated hydrocarbon chain having 2, 3, 4, 5, 6, 7, 8, 9, 10, 11 or 12 carbon atoms, wherein at least one double bond connects two carbon atoms at any point of the hydrocarbon chain, and wherein one hydrogen atom is removed from any carbon atom of the chain.

The term “straight or branched C₂-C₁₂ alkynyl” should be understood to encompass any straight or branched unsaturated hydrocarbon chain having 2, 3, 4, 5, 6, 7, 8, 9, 10, 11 or 12 carbon atoms, wherein at least one triple bond connects two carbon atoms at any point of the hydrocarbon chain, and wherein one hydrogen atom is removed from any carbon atom of the chain.

The term “straight or branched C₄-C₁₂ alkylene” should be understood to encompass any straight or branched saturated hydrocarbon chain having 4, 5, 6, 7, 8, 9, 10, 11 or 12 carbon atoms, wherein only sigma bonds connect between the atoms of the chain, and wherein two hydrogen atoms are removed from any two carbon atoms of the chain.

The term “straight or branched C₄-C₁₂ alkenylene” should be understood to encompass any straight or branched unsaturated hydrocarbon chain having 4, 5, 6, 7, 8, 9, 10, 11 or 12 carbon atoms, wherein at least one double bond connects two carbon atoms at any point of the hydrocarbon chain, and wherein two hydrogen atoms are removed from any two carbon atoms of the chain.

The term “straight or branched C₄-C₁₂ alkynylene” should be understood to encompass any straight or branched unsaturated hydrocarbon chain having 4, 5, 6, 7, 8, 9, 10, 11 or 12 carbon atoms, wherein at least one triple bond connects two carbon atoms at any point of the hydrocarbon chain, and wherein two hydrogen atoms are removed from any two carbon atoms of the chain.

The term “C₄-C₈ cycloalkylene” should be understood to encompass any saturated cyclic hydrocarbon ring having 4, 5, 6, 7, 8, carbon atoms, wherein only sigma bonds connect between the atoms of the ring, and wherein two hydrogen atoms are removed from any carbon atoms of the ring.

The term “C₄-C₈ cycloalkenylene” should be understood to encompass any cyclic unsaturated hydrocarbon ring having 4, 5, 6, 7, 8 carbon atoms, wherein at least one double bond connects two carbon atoms at any point of the hydrocarbon ring, and wherein two hydrogen atoms are removed from any two carbon atoms of the ring.

The term “C₄-C₈ cycloalkynylene” should be understood to encompass any cyclic unsaturated hydrocarbon ring having 4, 5, 6, 7, 8 carbon atoms, wherein at least one triple bond connects two carbon atoms at any point of the hydrocarbon ring, and wherein two hydrogen atoms are removed from any two carbon atoms of the ring.

As used herein, the term “arylene” refers to an aromatic ring system wherein two hydrogen atoms were removed thus having two open valencies for bonding. For example, a phenylene or a phenylene ring system fused to one or more aromatic rings to form, for example, derivatives of anthracene, phenanthrene, or napthalene ring systems.

The term “heteroarylene” refers to an aromatic ring system wherein at least one of the carbon atoms of the aromatic ring system is replaced by a heteroatom (N, O, P, S) and wherein two hydrogen atoms were removed, thus having two open valencies for bonding.

The present invention relates to pharmaceutical compositions comprising a compound of the subject invention in admixture with pharmaceutically acceptable auxiliaries, and optionally other therapeutic agents. The auxiliaries must be “acceptable” in the sense of being compatible with the other ingredients of the composition and not deleterious to the recipients thereof.

Pharmaceutical compositions include those suitable for oral, rectal, nasal, topical (including transdermal, buccal, and sublingual), vaginal or parenteral (including subcutaneous, intramuscular, intravenous, and intradermal) administration or administration via an implant. The compositions may be prepared by any method well-known in the art of pharmacy.

Such methods include the step of bringing in association compounds used in the invention or combinations thereof with any auxiliary agent. The auxiliary agent(s), also named accessory ingredient(s), include those conventional in the art, such as carriers, fillers, binders, diluents, disintegrants, lubricants, colorants, flavoring agents, anti-oxidants, and wetting agents.

Pharmaceutical compositions suitable for oral administration may be presented as discrete dosage units such as pills, tablets, dragées, or capsules, or as a powder or granules, or as a solution or suspension. The active ingredient may also be presented as a bolus or paste. The compositions can further be processed into a suppository or enema for rectal administration.

The invention further includes a pharmaceutical composition, as hereinbefore described, in combination with packaging material, including instructions for the use of the composition for a use as hereinbefore described.

For parenteral administration, suitable compositions include aqueous and non-aqueous sterile injection. The compositions may be presented in unit-dose or multi-dose containers, for example, sealed vials and ampoules, and may be stored in a freeze-dried (lyophilised) condition requiring only the addition of sterile liquid carrier, for example, water, prior to use. For transdermal administration, e.g., gels, patches, or sprays can be contemplated. Compositions or formulations suitable for pulmonary administration e.g., by nasal inhalation, include fine dust or mists, which may be generated by means of metered dose pressurized aerosols, nebulizers or insufflators.

The exact dose and regimen of administration of the composition will necessarily be dependent upon the therapeutic or nutritional effect to be achieved and may vary with the particular formula, the route of administration, and the age and condition of the individual subject to whom the composition is to be administered.

As used herein, the term “effective amount” means the amount of a drug or pharmaceutical composition that will elicit the biological or medical response of a tissue, system, animal, or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, slowing the progression of, or a decrease in the rate of advancement of a disease or disorder, condition or symptom. The term also includes within its scope amounts effective to enhance normal physiological function.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows the synthesis of compound of the invention ST-1948.

FIG. 2 shows the synthesis of compound of the invention ST-1949.

FIG. 3 shows the synthesis of compound of the invention ST-480.

FIG. 4 shows how ST-480 significantly decreases Huh-7 cells' survival. Huh-7 cells (2000 per well, in 96-wells plate) were treated with 100 μM of each compound. Cell viability was measured by MTT assay after 72 h. Asterisks represent a comparison to vehicle control. ****p<0.0001. N=3 biological repeats. Error bars represent SEM.

FIGS. 5A-5D show how ST-480 and CQ kill Huh-7 cells in a dose-dependent manner. (5A) Surviving curves in 200 mM paraquat (PQ). Worms were treated with ST-480 at the indicated concentrations or with the vehicle as a control for 48 h. Afterward, they were treated with 200 mM PQ, and their survival was measured after 3 and 6 h. N=6 biological repeats, P values are indicated (compared with the vehicle control). Bar graphs displaying the survival of Huh-7 cells in different doses of CQ (4B) and ST-480 (5C) —the experimental setup was similar to that described in FIG. 1. Asterisks represent a comparison to vehicle control. N=3 biological repeats. (5D) Bar graph showing the combined effect of ST-480 and CQ on Huh-7 cells' survival after 72 h. *p<0.05, ***p<0.001, ****p<0.0001. N=3 biological repeats. *Error bars represent SEM.

FIGS. 6A-6E show how ST-480 and CQ effectively kill Hep-3B cancer cells. Bar graphs displaying the effects of ST-480 (6A) and CQ (6B) and their combined effect 6C) on Hep-3B cells' survival after 72 h—the experimental setup was similar Asterisks represent a comparison to vehicle control (6A and 6B) or towards (the same CQ concentration under vehicle treatment conditions (5C). *p<0.05, **p<0.01, ***p<0.001, ****p<0.0001. N=3 biological repeats. (5D) Representative images show the state of Hep-3B cells treated as described in (6C). Scale bar: 200 μm. (5E) BEAS-2B cells are not sensitive to ST-480, ns-non-significance. N-3. Error bars represent SEM.

FIGS. 7A-7B show how ST-480 induces mitophagy. (7A) Colocalization of mitochondria and lysosome. Arrowheads indicate representative colocalization. The inset represents an eight-fold enlargement-scale bar: 50 μm. (7B) Colocalization quantification. N=3, ≥412 cells per treatment. *p<0.05, Error bars represent SEM.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Example 1: Synthesis of ST-1948

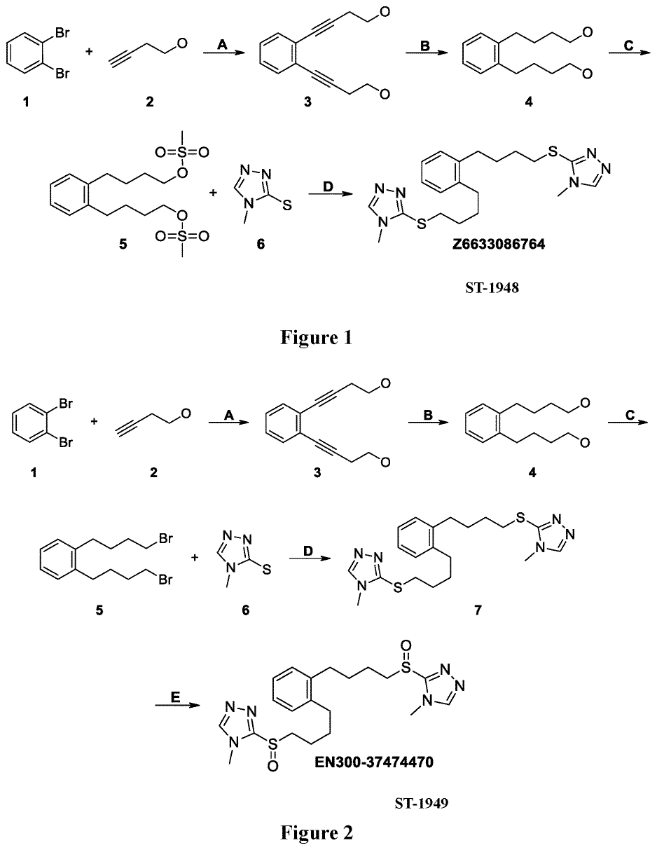

Synthesis of compound of the invention ST-1948 is shown in FIG. 1.

Step A: Reagent 1 (120 g, 1 eq), Reagent 2 (106.8 g, 3 eq), Et₃N (425 ml, 6 eq) were mixed in DMF (600 ml) and under Argon atmosphere CuI (4.8 g, 0.05 eq) and Tetrakis(triphenylphosphine palladium (0) (14.4 g, 0.025 eq) were added to the reaction mixture. The resulting solution was stirred at 85′C overnight. After that the reaction mixture was diluted with H₂O (600 ml), extracted with EtOAc (600 ml×2). The organic layer was separated, washed with H₂O (500 ml×2) and brine (500 ml), dried over anhydrous Na₂SO₄, filtered and evaporated under reduced pressure to give 161 g of crude Compound 3, which was purified with flash chromatography to afford 42 g of pure Compound 3.

Step B: To a solution of Reagent 3 (42 g, 1 eq) in MeOH (500 ml) was added 20% activated Pd/C (4.2 g) and the reaction mixture was hydrogenated in autoclave at 100 atm at 50′C 24 h. After that the reaction mixture was filtered and the resulting solution was concentrated under reduced pressure to afford 36.9 g of Compound 4.

Step C: To a solution of Reagent 4 (36.9 g, 1 eq) and Et₃N (69.3 ml, 3 eq) in dry DCM (400 ml) was added dropwise methanesulfonyl chloride (30.7 ml, 2.4 eq) at OC. The resulting solution was stirred at room temperature overnight. After that the reaction mixture was washed with water (2×400 ml). The organic layer was separated and concentrated under reduced pressure to afford 59 g of Compound 5.

Step D: To a solution of Reagent 5 (59 g, 1 eq) in MeOH (600 ml) at OC was added NaOH (15% solution in water, 4 eq). After 30 min Reagent 6 (71.7 g, 4 eq) was added to the reaction mixture at OC. The resulting solution was stirred at room temperature overnight. After that the reaction mixture was concentrated under reduced pressure, diluted with water and extracted with EtOAc (500 ml×2). The organic layer was separated, dried over anhydrous Na₂SO₄, filtered and evaporated under reduced pressure to give 54.3 g of crude desired product, which was purified by flash chromatography to afford 10 g of pure final compound Z6633086764 (ST-1948).

### Example 2: Synthesis of ST-1949

Synthesis of compound of the invention ST-1949 is shown in FIG. 2.

Step A: Reagent 1 (120 g, 1 eq), Reagent 2 (106.8 g, 3 eq), Et₃N (425 ml, 6 eq) were mixed in DMF (600 ml) and under Argon atmosphere CuI (4.8 g, 0.05 eq) and Tetrakis(triphenylphosphine palladium (0) (14.4 g, 0.025 eq) were added to the reaction mixture. The resulting solution was stirred at 85′C overnight. After that the reaction mixture was diluted with H₂O (600 ml), extracted with EtOAc (600 ml×2). The organic layer was separated, washed with H₂O (500 ml×2) and brine (500 ml), dried over anhydrous Na₂SO₄, filtered and evaporated under reduced pressure to give 161 g of crude Compound 3, which was purified with flash chromatography to afford 42 g of pure Compound 3.

Step B: To a solution of Reagent 3 (42 g, 1 eq) in MeOH (500 ml) was added 20% activated Pd/C (4.2 g) and the reaction mixture was hydrogenated in autoclave at 100 atm at 50 C 24 h. After that the reaction mixture was filtered and the resulting solution was concentrated under reduced pressure to afford 36.9 g of Compound 4.

Step C: To a solution of Reagent 4 (36.9 g, 1 eq) and Et₃N (69.3 ml, 3 eq) in dry DCM (400 ml) was added dropwise methanesulfonyl chloride (30.7 ml, 2.4 eq) at 0′C. The resulting solution was stirred at room temperature overnight. After that the reaction mixture was washed with water (2×400 ml). The organic layer was separated and concentrated under reduced pressure to afford 59 g of Compound 5.

Step D: To a solution of Reagent 5 (59 g, 1 eq) in MeOH (600 ml) at OC was added NaOH (15% solution in water, 4 eq). After 30 min, Reagent 6 (71.7 g, 4 eq) was added dropwise to the reaction mixture at OC. The resulting solution was stirred at room temperature overnight. After that, the reaction mixture was concentrated under reduced pressure, diluted with water, and extracted with EtOAc (500 ml×2). The organic layer was separated, dried over anhydrous Na₂SO₄, filtered, and evaporated under reduced pressure to give 54.3 g of the desired crude product, which was purified by flash chromatography to afford 17 g of pure compound 7.

Step E: To a solution of Reagent 7 (6 g, 1 eq) in DCM (120 ml) was added mCPBA (5.45 g, 85% purity, 2.2 eq) at OC. The resulting solution was stirred at room temperature overnight. The next day the mixture was washed with sat. aq. solution of K₂CO₃ (3×120 ml). The organic layer was separated and concentrated under reduced pressure to afford 9.9 g of crude product, which was purified by flash chromatography to result in 2.06 g of pure final compound EN300-37474470 (ST-1949).

### Example 3: Synthesis of ST-480

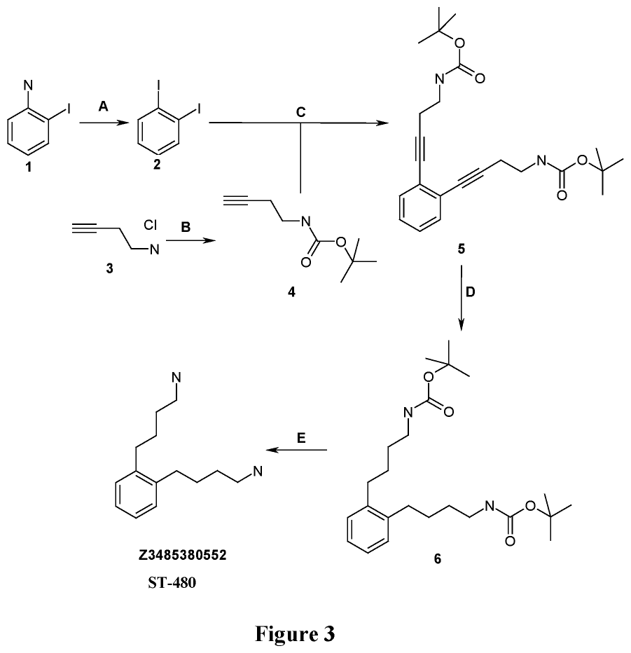

Synthesis of compound of the invention ST-480 is shown in FIG. 3.

Step A: Compound 1 (10 g, 45.7 mmol) was dissolved in acetone (100 mL), cooled to 0~5° C., and mixed with 37% concentrated hydrochloric acid. Aqueous sodium nitrite solution (3.62 g, 52.5 mmol) in water (15 mL) was added at 0~5° C., and the reaction mixture was stirred for 1~1.5 hours. Next, NaI (13.7 g, 91.3 mmol) was slowly added thereto. After that the reaction mixture was allowed to warm to room temperature and stirred for 30 minutes. The mixture was twice extracted with EtOAc (100 mL). The combined organic layer was washed with 10% aqueous solution of Na₂SO₃ (50 mL), dried over anhydrous Na₂SO₄, and the solvent was evaporated to afford 14 g of compound 2 (42.4 mmol, 93% yield).

Step B: Compound 3 (15 g, 142 mmol) and Boc₂O (31 g, 142 mmol) were mixed in dichloromethane (200 mL), and then triethylamine (30 mL) was added dropwise at 0° C. The resulting mixture was allowed to warm to r.t. and stirred overnight at the same temperature. After consumption of the starting material (controlled HNMR), the reaction mixture was transferred to separator funnel, washed with water (2×10 mL), dried over sodium sulfate, and evaporated in vacuo to give 21 g of compound 4 (124 mmol, 87% yield).

Step C: To a solution of compound 2 (13.75 g, 41.7 mmol), CuI (0.756 g, 4 mmol), and Pd(PPh₃)₄ (2.34 g, 2 mmol) in dry CH₃CN (75 mL), under an argon atmosphere, was added a solution of compound 4 (21 g, 124 mmol) in dry NEt₃ (10.3 mL). The reaction mixture was stirred under an argon atmosphere for 2 days. Next, it was diluted with CH₂Cl₂, washed by water, and concentrated under reduced pressure. The crude product was purified by preparative HPLC (hexane:EtOAc 15:1) to give 4.2 g of compound 5 (10.2 mmol, 25% yield) as a slightly brown solid.

Step D: Compound 5 (4.1 g, 9.94 mmol) was dissolved in methanol (60 mL) and treated with 10% Pd(OH)₂ on charcoal (0.4 g). The resulting mixture was hydrogenated at 20 bar and room temperature until the reaction was completed (TLC control). The catalyst was filtered off and the filtrate was evaporated to afford 4 g of compound 6 (9.51 mmol, 96% yield).

Step E: Compound 6 (4 g, 9.51 mmol) was dissolved in methanol (100 mL) and 4M HCl:dioxane (20 mL) at r.t. The resulting mixture was stirred overnight. Upon completion of the reaction (monitored by HNMR), it was evaporated to dryness to obtain 2.79 g od target compound Z3485380552 (ST-480) (9.51 mmol, 100% yield).

### Example 4: Evaluation of ST-1944 Cytotoxic Effect on Pancreatic, Liver, Lung and Glioma Human Cancer Cells, In Vitro Test System

Cell lines: PANC-1. Organism: Homo sapiens, human/Tissue: Pancreas; Duct/Disease: Epithelioid Carcinoma HUH7. Organism: Homo sapiens, human/Tissue: Liver/Disease: Carcinoma; Hepatocellular NCI-H69. Organism: Homo sapiens, human/Tissue: Lung/Disease: NSCL U87 MG. Organism: Homo sapiens, human/Tissue: Brain/Disease: Glioma

Test Item: ST-1944 at 50, 10, 2, and 0.4 μM. Vehicle: DMSO at 0.1, 0.02, 0.004, and 0.0008%.

### Experimental Design

PANC-1, HUH7, NCI-H69 and U87 MG cells are plated in one 96 well plate each, in their culture medium, at 7500 cells/well. Cells are allowed to attach for 16-24 hours at 37° C., 5% CO₂. Thereafter, culture medium is discarded, and fresh culture medium is added to the cells, supplemented with elevating ST-1944 concentrations: 0.4, 2, 10 and 50 μM, 3 wells of each concentration (triplicates). The cells are incubated another 72±2 hours at 37° C., 5% CO₂. At the end of incubation period, medium is discarded and 100 μL fresh culture medium is added to the cells along with 50 μL XTT reagent. The OD is measured in a plate reader once Vehicle treated cells are reach the range of 0.5-1.5 OD at 450 nm wavelength.

### Materials and Formulations

PANC-1 and HUH7culture medium: DMEM Medium (4.5 g/L glucose), supplemented with 10% FBS (heat inactivated), 2 mM L-Glutamine, and 1% of Pen/Strep Solution. U87 MG and NCI-H69 culture medium: EMEM Medium, supplemented with 10% FBS (heat inactivated), 2 mM L-Glutamine, and 1% of Pen/Strep Solution. Test Items: Test Item ST-1944 solution is prepared as 50 mM stock in DMSO and diluted in culture medium 1:1,000, 1:5,000, 1:25,000, and 1:125,000 yielding 50, 10, 2, and 0.4 μM working concentrations with the cells, respectively. Vehicle: DMSO is diluted in culture medium 1:1,000, 1:5,000, 1:25,000, and 1:125,000 yielding 0.1, 0.02, 0.004, and 0.0008% working concentrations with the cells, respectively.

### Results Processing:

The mean OD of each of Test Item (ST-1944) concentration was divided by the mean OD of the corresponding Vehicle (DMSO) concentration to yield % inhibition. The IC₅₀ value (ST-1944 concentration that inhibits cell proliferation by 50%) was then calculated by interpolation of the % inhibition data, for each cell line.

TABLE 1
IC₅₀ of human cancer cells' inhibition of proliferation by ST-1944

| Cell line | Tumor type | IC₅₀ (μM) |
|---|---|---|
| HUH7 | Liver cancer (HCC) | 2 |
| Panc-1 | Pancreatic cancer | 1 |
| NCI-H69 | Lung cancer (NSCLC) | 10 |
| U87 MG | Glioma (GBM) | 10 |

### Example 5: Anticancer Activity of ST-480

Liver cancer is the deadliest cancer globally, and it is the sole one among the five deadliest types of cancer to have an increase in annual cases. Currently, surgery is the only potential cure for liver cancer, while chemotherapy, immunotherapy, and radiotherapy mainly provide symptom relief. Therefore, there is an urgent need to develop effective therapies for liver cancer. Mitochondrial autophagy, or mitophagy, is a process that removes damaged mitochondria from the mitochondrial network, and it appears to play a key role in liver cancer development. In fact, enhancement of mitophagy through natural or synthetic compounds such as concanavalin A, melatonin, and Adriamycin has been shown to suppress liver cancer cell growth. ST-480, was found to be able to induce robust mitophagy in liver cancer cell lines and selectively kill them. Additionally, it was discovered that the antimalaria drug chloroquine (CQ) enhances ST-480's anticancer activity.

### Results

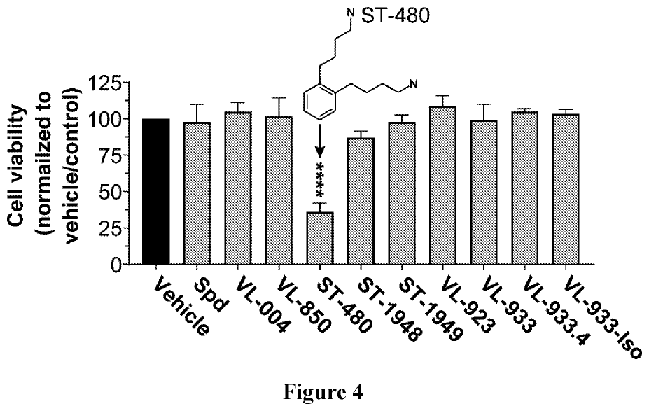

ST-480, is one of the diamine compounds of the invention that inhibits liver cancer cell viability. Several studies show that mitophagy activation can promote liver cancer cell death activation. Therefore, the activity of the polyamine compound of the invention in liver cancer therapy. To explore this, the inventors screened the effect of eight new compounds. In addition, the effect of two compounds in the context of lifespan, health-span, and proteotoxicity, i.e., the diamines VL-004 and VL-850 were examined, and the natural polyamine spermidine (Spd). Huh-7 cells (derived from hepatocellular carcinoma of a 57-year-old male) were exposed to 100 μM of each compound and examined their survival after 72 h using an MTT assay. ST-480, significantly decreased Huh-7 cell survival (FIG. 4).

Chloroquine (CQ) enhances ST-480 cytotoxicity. ST-480 and the compounds VL-004 and VL-850 confer resistance to oxidative stress in the nematode C. elegans (FIG. 4A); it is important to note that ST-480 did not show any toxicity to worms at the concentrations that were tested (up to 250 μM).

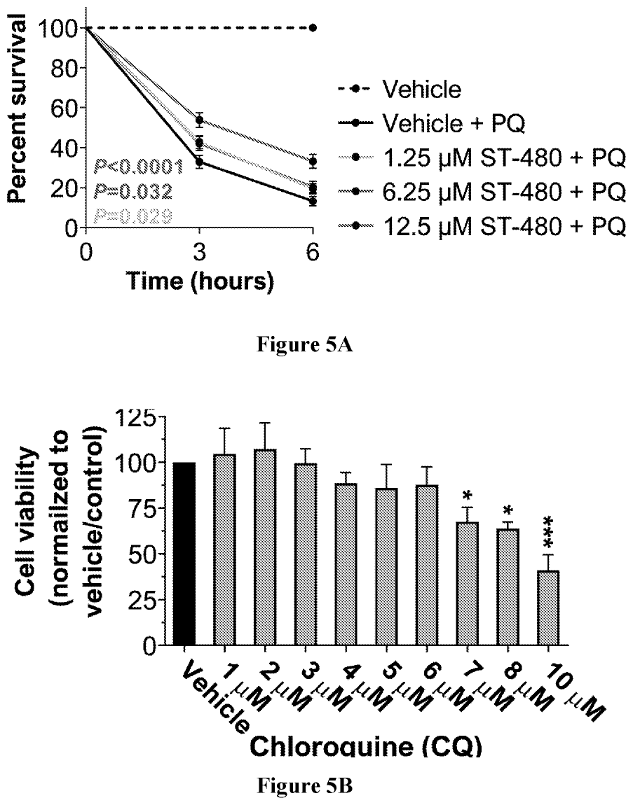

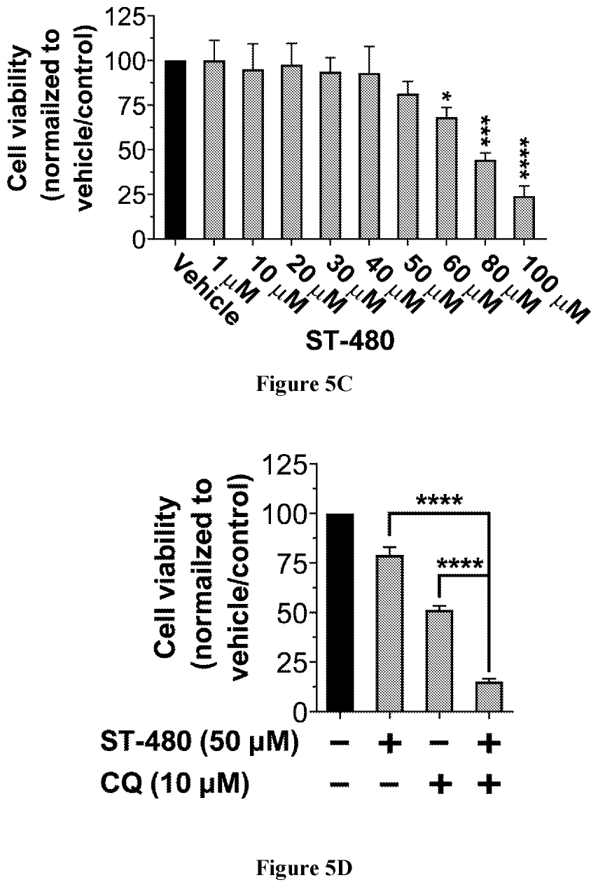

The working model suggests that ST-480 kills cancer cells by disturbing autophagy flux and increasing apoptosis. Chloroquine (CQ) has been used safely to treat malaria and to suppress autophagic flux. Moreover, as indicated above, it induces DNA damage and apoptosis in cancer cells. The effect of different CQ concentrations on Huh-7 cells' survival after 72 h were tested. In parallel, dose-response experiments testing ST-480 toxicity in these cells were performed. CQ significantly decreased cells' survival at 7, 8, and 10 μM and ST-480 at 60, 80, and 100 μM (FIG. 4B, 4C). Strikingly, the combined effect of 50 μM ST-480 and 10 μM CQ was greater than the sum of both (FIG. 5D).

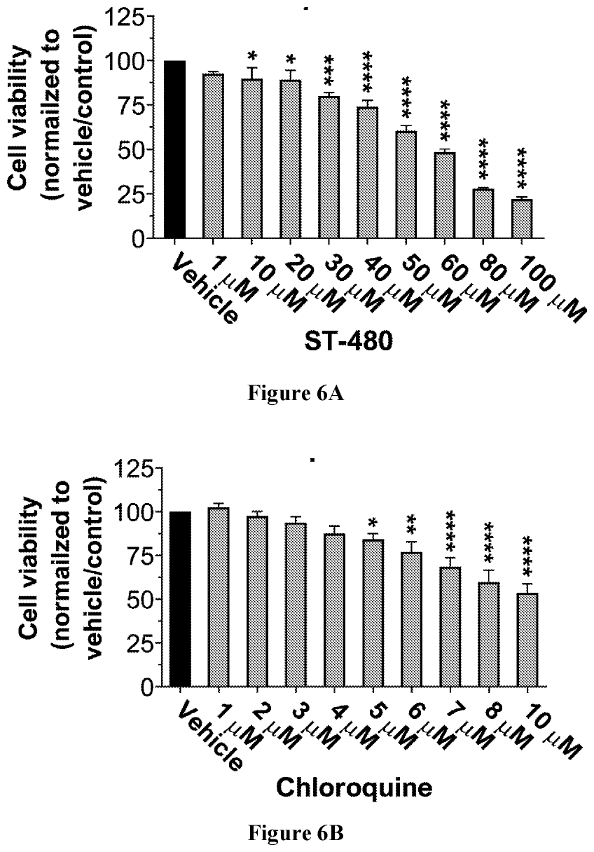

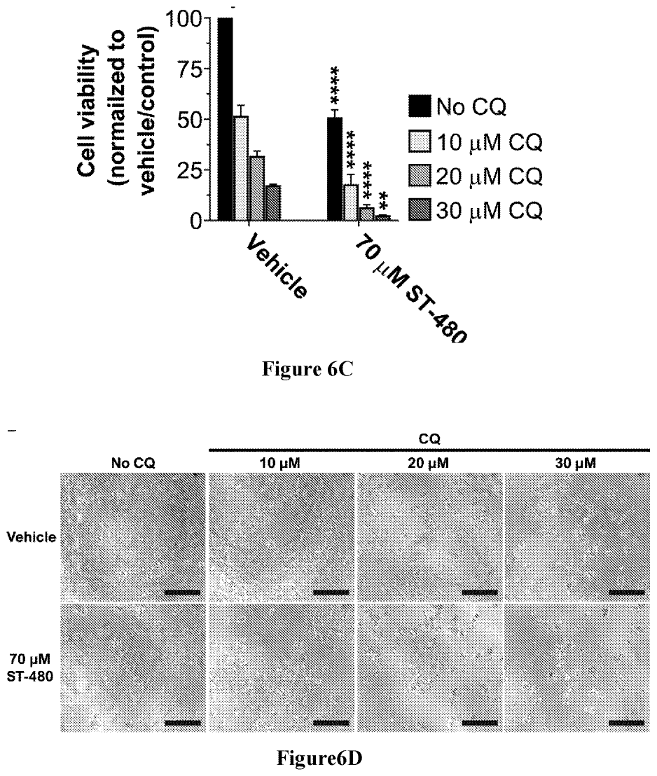

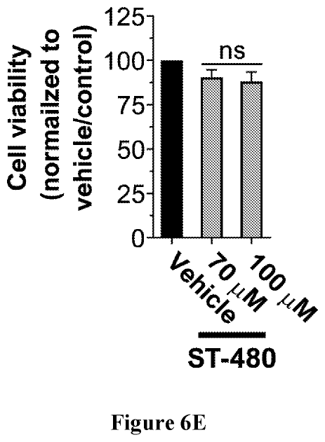

To strengthen these conclusions, the impact of ST-480 and CQ was tested on another liver cancer cell line, namely Hep-3B′ the Hep-3B cell line originated from an 8-year-old black male with primary hepatocellular carcinoma (HCC). Hep-3B cells were sensitive to both ST-480 and CQ in a dose-dependent manner (FIG. 6A, 6B). In addition, the combined effect of ST-480 and CQ on Hep-3B survival was tested. Like the Huh-7 results, increased toxicity by the joint treatment was observed (FIG. 6C, 6D), further supporting the conclusion that autophagy inhibition enhances the cancer-killing activity of ST-480. In this respect, it should be emphasized that ST-480 was not toxic to non-cancer cells. BEAS-2B cells were exposed to 70 UM and 100 μM ST-480 for 72 h and did not observe a significant cell death (FIG. 6E), suggesting that ST-480 selectively kills cancer cells.

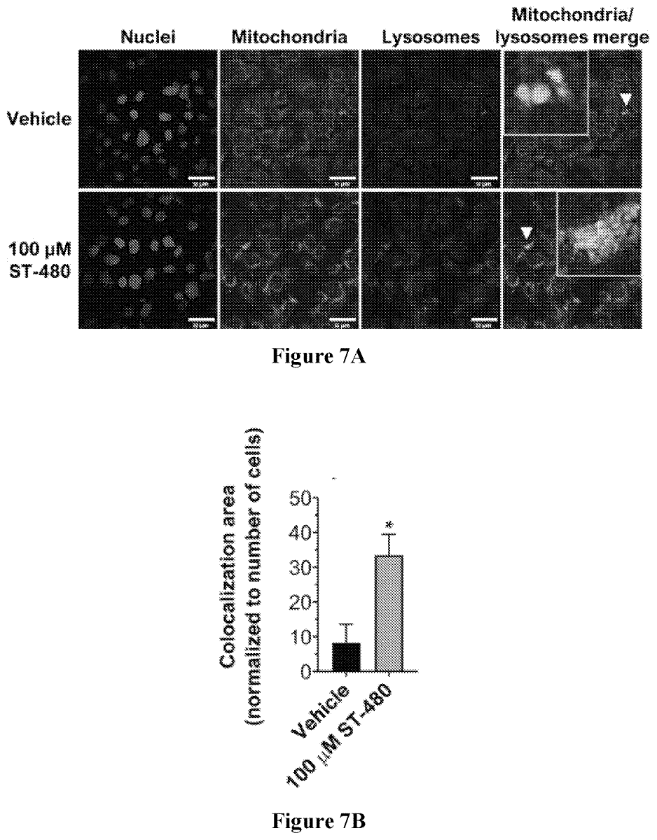

ST-480 induces mitophagy in Hep-3B cells. To explore whether ST-480 induces mitophagy, Hep-3B cells were exposed to 100 μM ST-480 or vehicle control for 6 h. To measure mitophagy, the colocalization of mitochondria and lysosomes were quantified using specific mitochondrial and lysosomal dyes. In addition, nuclei were stained using a DNA dye—For this, the Cytopainter ab139487 kit, was used. ST-480 induces mitophagy in a significant manner (FIG. 7).

1. A compound having a general formula (I) 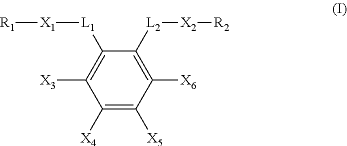
wherein each of R₁ and R₂ is independently selected from —C(═NR₃)NR₄R₅, —NR₆R₇, —N⁺R₈R₉R₁₀, —NR₁₁C(═N)NR₁₂R₁₃, —NR₁₈NR₁₉R₂₀, —NR₁₄C(═N)—NR₁₅—C(═N)—NR₁₆R₁₇=N—R₂₁, 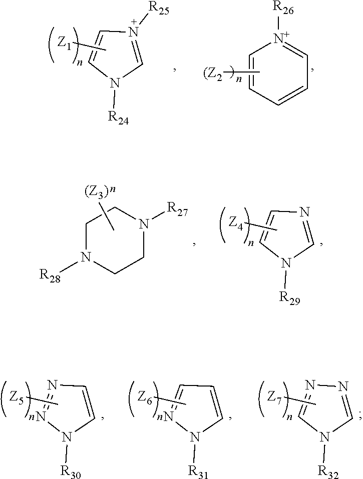
wherein
each of R₃-R₃₂ is independently selected from H, straight or branched C₁-C₁₂ alkyl, straight or branched C₂-C₁₂ alkenyl, straight or branched C₂-C₁₂ alkynyl, phenyl, —OH and any combinations thereof;
each of L₁ and L₂ is independently selected from straight or branched C₄-C₁₂ alkylene, straight or branched C₄-C₁₂ alkenylene, straight or branched C₄-C₁₂ alkynylene;
each L₁ and L₂ is independently optionally interrupted by at least one of C₄-C₈ cycloalkylene, C₄-C₈ cycloalkenylene, C₄-C₈ cycloalkynylene, arylene, heteroarylene, heteroatom and any combinations thereof;
each of L₁ and L₂ is independently optionally substituted with at least one of halogen and any combinations thereof;
each of X₁ and X₂ is independently selected from null, —O—, —S—, —S(═O)—, —S(═O)₂—;
each of Z₁-Z₇ is independently a halogen; wherein n is independently an integer selected from 0-8;
each of X₃—X₆ is independently selected from H, halogen, astatine, tennessine and any combinations thereof.

2. A compound according to claim 1, wherein each of L₁ and L₂ is independently straight or branched C₄-C₁₂ alkylene.

3. A compound according to claim 1, wherein each of L₁ and L₂ is independently interrupted by at least one of C₄-C₈ cycloalkylene, C₄-C₈ cycloalkenylene, C₄-C₈ cycloalkynylene, aryl, heteroaryl, heteroatom and any combinations thereof.

4. (canceled)

5. (canceled)

6. (canceled)

7. (canceled)

8. (canceled)

9. (canceled)

10. (canceled)

11. (canceled)

12. (canceled)

13. (canceled)

14. A compound according to claim 1, wherein each of X₁ and X₂ is independently selected from —O—, —S—, —S(═O)—, —S(═O)₂.

15. (canceled)

16. (canceled)

17. (canceled)

18. (canceled)

19. (canceled)

20. (canceled)

21. A compound according to claim 1, wherein R₁ and R₂ are each —C(═NR₃)NR₄R₅.

22. A compound according to claim 1, wherein R₁ and R₂ are each selected from —NR₆R₇ and —N⁺R₈R₉R₁₀.

23. A compound according to claim 1, wherein R₁ and R₂ are each selected from —NR₁₁C(═N)NR₁₂R₁₃ and —NR₁₄C(═N)—NR₁₅—C(═N)—NR₁₆R₁₇.

24. A compound according to claim 1, wherein R₁ and R₂ are each —NR₁₈NR₁₉R₂₀.

25. A compound according to claim 1, wherein R₁ and R₂ are each ═N—R₂₁.

26. (canceled)

27. (canceled)

28. (canceled)

29. (canceled)

30. (canceled)

31. (canceled)

32. (canceled)

33. A compound according to claim 1, wherein X₁ and X₂ are each independently selected from —O—, —S— and any combinations thereof.

34. A compound according to claim 1, wherein X₁ and X₂ are each independently selected —S(═O)—, —S(═O)₂— and any combinations thereof.

35. A compound according to claim 1, wherein each Z₁, Z₂, Z₃, Z₄, Z₅, Z₆ and Z₇ is a halogen.

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. A method of slowing the progression of or preventing a condition or a disease associated with cell degeneration, said method comprising administering to said subject an effective dose of a compound according to any one of claim 1.

50. A method of claim 49, said method comprising administering to said subject, in addition, an effective dose of autophagy inhibitor agent.

51. (canceled)

52. (canceled)

53. (canceled)

54. (canceled)

55. (canceled)

56. A method according to claim 49, wherein said condition or a disease associated with cell degeneration is selected from Parkinson's disease, Alzheimer's disease, dementia, congestive heart failure, sarcopenia, type 2 diabetes, age-related macular degeneration (AMD), atherosclerosis, cardiovascular diseases, cancer, liver diseases, pancreatic diseases, ocular diseases, arthritis, cataracts, osteoporosis, hypertension, fibrosis, including lung-fibrosis, and any combinations thereof.

57. (canceled)

58. (canceled)

59. (canceled)
